(19) United States
(12) Patent Application Publication
(10) Pub. No.: US 2012/0093771 A1
(43) Pub. Date: Apr. 19, 2012

(54) METHOD FOR PREPARING A HARDENED CALCIUM SULFATE DIHYDRATE BLOCK AND USE THEREOF

(75) Inventors: Jiin-Huey Chern LIN, Winnetka, IL (US); Chien-Ping Ju, Kansas, MO (US)

(73) Assignee: NATIONAL CHENG KUNG UNIVERSITY, Tainan City (TW)

(21) Appl. No.: 12/907,104

(22) Filed: Oct. 19, 2010

(51) Int. Cl. A61K 38/18; A61K 47/04; A61P 19/00; A61F 2/28; B29C 39/00; B05D 3/00; A61C 5/02; A61K 35/00; C04B 12/02

(52) U.S. Cl. 424/93.1; 514/7.6; 514/769; 106/35; 106/690; 264/299; 264/333; 427/2.26; 433/224; 623/23.62

(57) ABSTRACT

The present invention provides a technique for prolonging the working time and setting time of a calcium sulfate hemihydrate paste by mixing calcium sulfate hemihydrate powder with an aqueous solution containing phosphate ions, so that the paste is suitable for operation and the resultant hardened calcium sulfate dihydrate block from the paste has an improved mechanical strength.

### FIELD OF THE INVENTION

The present invention is related to a method for preparing a hardened calcium sulfate dihydrate block from a paste of calcium sulfate hemihydrate powder and an aqueous solution, and related to methods for using the hardened calcium sulfate dihydrate in an orthopedic treatment or a dental treatment such as a root canal treatment.

### BACKGROUND OF THE INVENTION

For many orthopedic applications, calcium sulfate dihydrate is not an ideal implant material due to its insufficient compressive strength and/or a dissolution rate that is too high in comparison with the calcium phosphate implant material. Furthermore, one major drawback of calcium sulfate hemihydrate bone cement is that it conventionally has a working time and setting time which are not sufficiently long for operation. Accordingly, researchers have been trying various approaches to (but still are not successful to) develop calcium sulfate hemihydrate bone cement with suitable working time and setting time and with a desired compressive strength of the resultant hardened calcium sulfate dihydrate block.

### SUMMARY OF THE INVENTION

A primary object of the present invention is to provide a bone cement formula comprising calcium sulfate hemihydrate powder as a major powder portion and an aqueous solution, so that a paste formed from said formula is suitable for operation and the resultant hardened calcium sulfate dihydrate block from the paste has an improved mechanical strength.

Another object of the present invention is to provide a method for preparing a hardened calcium sulfate dihydrate block by using the bone cement formula of the present invention.

Another object of the present invention is to provide methods for treating a subject who needs an orthopedic treatment or a root canal treatment.

Preferred embodiments of the present invention include (but not limited to) the following items:

1. A method for preparing a hardened calcium sulfate dihydrate block comprising mixing calcium sulfate hemihydrate powder with an aqueous solution containing phosphate ions so as to form a paste, wherein the aqueous solution has a pH value lower than 10.

2. The method of Item 1 wherein there in no alkaline compound being added to the aqueous solution, the calcium sulfate hemihydrate power or the paste thereof to adjust pH values thereof.

3. The method of Item 1 wherein the aqueous solution has a temperature lower than 50° C. before said mixing.

4. The method of Item 3 wherein said mixing is conducted at a temperature lower than 50° C.

5. The method of Item 3 wherein the paste of the power and the aqueous solution has a temperature lower than 50° C.

6. The method of Item 1 wherein there is no heating being applied to the aqueous solution, the calcium sulfate hemihydrate power or the paste thereof to elevate temperatures thereof to equal to or greater than 50° C.

7. The method of Item 6 wherein the aqueous solution has a concentration of the phosphate ions lower than 1.0 M.

8. The method of Item 7, wherein the concentration is of 0.01 M to 0.5 M.

9. The method of Item 1 wherein the mixing is conducted with a liquid to powder ratio of 0.20 cc/g to 0.60 cc/g.

10. The method of Item 9, wherein the liquid to powder ratio is 0.30 cc/g to 0.50 cc/g.

11. The method of Item 1, wherein said aqueous solution is an aqueous solution of (NH₄)₃PO₄, (NH₄)₂HPO₄, NH₄H₂PO₄, K₃PO₄, K₂HPO₄, KH₂PO₄, Na₃PO₄, Na₂HPO₄, NaH₂PO₄, H₃PO₄, or a mixture thereof.

12. The method of Item 11, wherein said aqueous solution is an aqueous solution of (NH₄)₃PO₄, (NH₄)₂HPO₄, NH₄H₂PO₄, or a mixture thereof.

13. The method of Item 1 where in said aqueous solution or said calcium sulfate hemihydrate powder further comprises living cells, a growth factor or a drug.

14. The method of Item 1, wherein said calcium sulfate hemihydrate powder is an alpha-type calcium sulfate hemihydrate powder.

15. The method of Item 1 further comprising introducing the paste to a hole or cavity and letting the paste become set in-situ to form a block of hardened calcium sulfate dihydrate in the hole or cavity.

16. The method of Item 1 further comprising shaping the paste in a mold, and removing the mold to form a block of hardened calcium sulfate dihydrate.

17. The method of Item 16 further comprising pressurizing said paste in said mold before said paste becomes set to remove a portion of liquid from said paste, so that a liquid to powder ratio of said paste decreases.

18. The method of Item 17, wherein the pressure applied to the paste in the mold is form about 1 MPa to 500 MPa, preferably from 100 MPa to 500 MPa.

19. The method of Item 16 further comprising impregnating the block with an impregnating liquid for a period of time, so that a compressive strength of the resulting impregnated block removed from the impregnating liquid is increased compared to that of said block without said impregnating treatment.

20. The method of Item 19 wherein the impregnating liquid is a phosphate-containing solution.

21. The method of Item 20, wherein the phosphate-containing solution is an aqueous solution of (NH₄)₃PO₄, (NH₄)₂HPO₄, NH₄H₂PO₄, K₃PO₄, K₂HPO₄, KH₂PO₄, Na₃PO₄, Na₂HPO₄, NaH₂PO₄, or H₃PO₄.

22. The method of Item 20, wherein said phosphate-containing solution has a phosphate concentration from about 0.1 M to about 6 M, preferably from about 1 M to about 3 M.

23. The method of Item 20, wherein said impregnating is conducted at a temperature of about 0° C.

24. The method of Item 1 further comprising mixing a pore-forming agent with the powder or with the paste; shaping the paste in a mold; removing the mold to form a block of hardened calcium sulfate dihydrate with the pore-forming agent embedded therein; and immersing said block of hardened calcium sulfate dihydrate with the pore-forming agent embedded therein in an immersing liquid to dissolve said pore-forming agent in the immersing liquid, creating pores therein, so that a porous block is formed.

25. The method of Item 24, wherein the pore forming agent is selected from the group consisting of LiCl, KCl, NaCl, MgCl₂, CaCl₂, NaIO₃, KI, Na₃PO₄, K₃PO₄, Na₂CO₃, amino acid-sodium salt, amino acid-potassium salt, glucose, polysaccharide, fatty acid-sodium salt, fatty acid-potassium salt, potassium bitartrate (KHC₄H₄O₆), potassium carbonate, potassium gluconate (KC₆H₁₁O₇), potassium-sodium tartrate (KNaC₄H₄O₆.4H₂O), potassium sulfate (K₂SO₄), sodium sulfate, sodium lactate and mannitol.

26. The method of Item 24, wherein the immersing liquid is an acidic aqueous solution, a basic aqueous solution, a physiological solution, an organic solvent, or water.

27. The method of Item 26, wherein the immersing liquid is a phosphate-containing solution having a phosphate concentration from about 0.1 M to about 6 M, preferably from about 1 M to about 3 M.

28. The method of Item 27, wherein the phosphate-containing solution is an aqueous solution of (NH₄)₃PO₄, (NH₄)₂HPO₄, NH₄H₂PO₄, K₃PO₄, K₂HPO₄, KH₂PO₄, Na₃PO₄, Na₂HPO₄, NaH₂PO₄, or H₃PO₄.

29. The method of Item 27, wherein the immersing liquid is water.

30. The method of Item 24 further comprising impregnating the porous block with an impregnating liquid for a period of time, so that a compressive strength of the resulting impregnated porous block removed from the impregnating liquid is increased compared to that of said porous block without said impregnating treatment.

31. The method of Item 30 wherein the impregnating liquid is a phosphate-containing solution.

32. The method of Item 31, wherein the phosphate-containing solution is an aqueous solution of (NH₄)₃PO₄, (NH₄)₂HPO₄, NH₄H₂PO₄, K₃PO₄, K₂HPO₄, KH₂PO₄, Na₃PO₄, Na₂HPO₄, NaH₂PO₄, or H₃PO₄.

33. The method of Item 31, wherein said phosphate-containing solution has a phosphate concentration from about 0.1 M to about 6 M, preferably from about 1 M to about 3 M.

34. The method of Item 27 further comprising impregnating the porous block in a suspension of living cells or a solution of growth factor or drug to deposit the living cells, the growth factor or drug onto the porous block.

35. The method of Item 24, wherein the porous block has a porosity of 50-90 vol %.

36. A method for treating a subject comprising mixing calcium sulfate hemihydrate powder with an aqueous solution containing phosphate ions so as to form a paste; and filling a hole or cavity in a bone of said subject with said paste which set hard in the hole or cavity in need of said treatment.

37. The method of Item 36 wherein said treatment is an orthopedic treatment or a dental treatment.

38. A method for treating a subject comprising mixing calcium sulfate hemihydrate powder with an aqueous solution containing phosphate ions so as to form a paste; forming a block of hardened calcium sulfate dihydrate from said paste; and implanting said block in said subject in need of said treatment.

39. The method of Item 38 wherein said treatment is an orthopedic treatment or a dental treatment.

40. The method of Item 39 wherein said implanting comprises breaking up the block into pellets and filling a hole or cavity in a bone of said subject with said pellets.

41. The method of Item 40 wherein said formation of the block of hardened calcium sulfate dihydrate comprising shaping the paste in a mold, and removing the mold to form a block of hardened calcium sulfate dihydrate.

42. The method of Item 41 wherein said formation of the block of hardened calcium sulfate dihydrate further comprising pressurizing said paste in said mold before said paste becomes set to remove a portion of liquid from said paste, so that a liquid to powder ratio of said paste decreases.

43. The method of Item 42, wherein the pressure applied to the paste in the mold is form about 1 MPa to 500 MPa, preferably from 100 MPa to 500 MPa.

44. The method of Item 41, wherein said formation of the block of hardened calcium sulfate dihydrate further comprising impregnating the block with an impregnating liquid for a period of time, so that a compressive strength of the resulting impregnated block removed from the impregnating liquid is increased compared to that of said block without said impregnating treatment.

45. The method of Item 44 wherein the impregnating liquid is a phosphate-containing solution.

46. The method of Item 45, wherein the phosphate-containing solution is an aqueous solution of (NH₄)₃PO₄, (NH₄)₂HPO₄, NH₄H₂PO₄, K₃PO₄, K₂HPO₄, KH₂PO₄, Na₃PO₄, Na₂HPO₄, NaH₂PO₄, or H₃PO₄.

47. The method of Item 45, wherein said phosphate-containing solution has a phosphate concentration from about 0.1 M to about 6 M, preferably from about 1 M to about 3 M.

48. The method of Item 45, wherein said impregnating is conducted at a temperature of about 0° C.

49. The method of Item 38 further comprising mixing a pore-forming agent with the powder or with the paste, wherein said formation of the block of hardened calcium sulfate dihydrate comprises shaping the paste in a mold; removing the mold to form a block of hardened calcium sulfate dihydrate with the pore-forming agent embedded therein; and immersing said block of hardened calcium sulfate dihydrate with the pore-forming agent embedded therein in an immersing liquid to dissolve said pore-forming agent in the immersing liquid, creating pores therein, so that a porous block is formed, and thus said implanting comprises implanting said porous block in said subject in need of said treatment.

50. The method of Item 49 wherein said implanting comprises breaking up the porous block into pellets and filling a hole or cavity in a bone of said subject with said pellets.

51. The method of Item 50, wherein the pore forming agent is selected from the group consisting of LiCl, KCl, NaCl, MgCl₂, CaCl₂, NaIO₃, KI, Na₃PO₄, K₃PO₄, Na₂CO₃, amino acid-sodium salt, amino acid-potassium salt, glucose, polysaccharide, fatty acid-sodium salt, fatty acid-potassium salt, potassium bitartrate (KHC₄H₄O₆), potassium carbonate, potassium gluconate (KC₆H₁₁O₇), potassium-sodium tartrate (KNaC₄H₄O₆.4H₂O), potassium sulfate (K₂SO₄), sodium sulfate, sodium lactate and mannitol.

52. The method of Item 50, wherein the immersing liquid is an acidic aqueous solution, a basic aqueous solution, a physiological solution, an organic solvent, or water.

53. The method of Item 52, wherein the immersing liquid is a phosphate-containing solution having a phosphate concentration from about 0.1 M to about 6 M, preferably from about 1 M to about 3 M.

54. The method of Item 53, wherein the phosphate-containing solution is an aqueous solution of (NH₄)₃PO₄, (NH₄)₂HPO₄, NH₄H₂PO₄, K₃PO₄, K₂HPO₄, KH₂PO₄, Na₃PO₄, Na₂HPO₄, NaH₂PO₄, or H₃PO₄.

55. The method of Item 53, wherein said immersing liquid is water.

56. The method of Item 50 further comprising impregnating the porous block with an impregnating liquid for a period of time, so that a compressive strength of the resulting impregnated porous block removed from the impregnating liquid is increased compared to that of said porous block without said impregnating treatment.

57. The method of Item 56 wherein the impregnating liquid is a phosphate-containing solution.

58. The method of Item 57, wherein the phosphate-containing solution is an aqueous solution of (NH₄)₃PO₄, (NH₄)₂HPO₄, NH₄H₂PO₄, K₃PO₄, K₂HPO₄, KH₂PO₄, Na₃PO₄, Na₂HPO₄, NaH₂PO₄, or H₃PO₄.

59. The method of Item 57, wherein said phosphate-containing solution has a phosphate concentration from about 0.1 M to about 6 M, preferably from about 1 M to about 3 M.

60. The method of Item 49 further comprising impregnating the porous block in a suspension of living cells or a solution of growth factor or drug to deposit the living cells, the growth factor or drug onto the porous block.

61. The method of Item 49, wherein the porous block has a porosity of 50-90 vol %.

### DETAILED DESCRIPTION OF THE INVENTION

The present invention provides a technique for prolonging the working time and setting time of a calcium sulfate hemihydrate paste by mixing calcium sulfate hemihydrate powder with an aqueous solution containing phosphate ions, so that the paste is suitable for operation and the resultant hardened calcium sulfate dihydrate block from the paste also has an improved mechanical strength. A bone cement formula provided according to the present invention is composed of calcium sulfate hemihydrate powder, an aqueous solution containing phosphate ions, and one or more optionally functional components such as living cells, a growth factor, a drug or a pore-forming agent.

The present invention will be better understood through the following examples which are illustrative only and are not intended to be in any way limiting.

### EXAMPLES

Chemicals used in the examples:

| Chemical | Formula | Supplier |
|---|---|---|
| calcium sulfate | CaSO₄ 0.5H₂O | Showa, Tokyo, Japan |
| hemihydrate (CSH) | | |
| calcium sulfate | CaSO₄ 2H₂O | Panreac, E.U. |
| Dihydrate (CSH) | | |
| Calcium sulfate | CaSO₄ | Showa, Tokyo, Japan |
| Diammonium hydrogen | (NH₄)₂HPO₄ | Showa, Tokyo, Japan |
| phosphate | | |
| Disodium hydrogen | Na₂HPO₄ | Showa, Tokyo, Japan |
| phosphate | | |
| phosphoric acid | H₃PO₄ | Showa, Tokyo, Japan |
| hydrochloric acid | HCl | Nihon Shiyaka, Osaka, |
| | | Japan |
| sodium hydroxide | NaOH | Showa, Tokyo, Japan |
| Dipotassium hydrogen | K₂HPO₄ | Katayama, Osaka, |
| phosphate | | Japan |
| Diammonium | (NH₄) H₂PO₄ | Showa, Tokyo, Japan |
| dihydrogen phosphate | | |
| Sodium dihydrogen | NaH₂PO₄ | Showa, Tokyo, Japan |
| phosphate | | |
| Potassium dihydrogen | KH₂PO₄ | Icatayama, Tokyo, |
| phosphate | | Japan |
| Tartaric acid | C₂H₂(OH)₂(COOH)₂ | Katayam, Osaka, Japan |
| Malic acid | C₂H₃(OH)(COOH)₂ | Pantreac, Barcelona, |
| | | Spain |
| Potassium chloride | KCl | Showa, Tokyo, Japan |
| Sodium chloride | NaCl | Katayam, Osaka, Japan |
| Potassium dihydrogen | KH₂PO₄ | Showa, Tokyo, Japan |
| phosphate | | |
| Sodium hydrogen | NaHCO₃ | Atayam, Osaka, Japan |
| carbonate | | |

### Preparation of a Calcium Sulfate Cement Paste

The calcium sulfate cement paste was prepared by mixing appropriate amounts of CSH powder and hardening solution (for example, diammonium dihydrogen phosphate or dipotassium hydrogen phosphate) with a desirable liquid/powder ratio (for example, 0.35 cc/g).

### Preparation of a Calcium Sulfate Dense Block Article

Appropriate amounts of CSH powder and hardening solution were uniformly mixed in a ball miller at a desirable L/P ratio to form a calcium sulfate cement paste.

Prior to being fully hardened, the paste was placed in a mold under a desirable pressure (for example, 450 Kgf) to squeeze a portion of the hardening solution out of the paste to form a hardened dense block. After being removed from the mold, one group of the hardened samples was placed in a moisture-proof container for 1 day. Another group of samples was further impregnated in an impregnation solution (for example, (NH₄)₂HPO₄ or K₂HPO₄) at a desirable temperature (for example, 37° C.) for a period of time (for example, 1 day), followed by drying in an oven at 50° C. for 1 day.

### Preparation of a Calcium Sulfate Porous Block Article

Appropriate amounts of CSH powder, pore-forming particles (for example KCl) and hardening solution were uniformly mixed with a desirable KCl/CSH ratio (for example 1:1 or 1.5:1 by weight) and a desirable liquid/powder ratio (for example, 0.35 cc/g) to form a KCl/CSH cement paste.

Prior to being fully hardened, the KCl/CSH cement paste was placed in a mold under a desirable pressure (for example, 450 Kgf) to squeeze a portion of the hardening solution out of the paste to for a hardened dense block. After being removed from the mold, the hardened dense block sample was immersed in de-ionized water for a period of time (for example, 3 days) to allow the pore-forming particles to be washed out of the dense block to form a calcium sulfate porous block, followed by drying in an oven at 50° C. for 1 day.

One group of the porous block samples was further impregnated in an impregnation solution (e.g. (NH₄)₂HPO₄ or K₂HPO₄) at a desirable temperature (for example 37° C. or 4° C.) for a period of time to allow the strength of the porous block to increase, followed by drying in an oven at 50° C. for 1 day. To remove the residual impregnation solution from inside the pores, the impregnated porous samples were rinsed in de-ionized water for a period of time (for example, 3 days).

Another group of samples was further treated by immersion in CaCl₂ solution for a period of time (for example, 3 days) to further enhance the strength of the porous samples.

### Compressive Strength (CS) Testing

To measure the CS of a hardened cement, after mixing for 1 min, the cement paste was packed in a 6 mm diameter, 12 mm deep cylindrical stainless steel mold under a pressure of 0.7 MPa for 30 min. After being removed from the mold, the hardened cement samples were immersed in Hanks' physiological solution which was maintained at 37° C. and agitated daily to help maintain uniform ion concentrations. After immersion, samples were removed from the solution for CS testing while samples are still wet. The CS testing was conducted using a desk-top mechanical tester (Shimadzu AGS-500D, Tokyo, Japan) at a crosshead speed of 1.0 mm/min. The test method is according to ASTM 451-99a method.

### Working Time (WT)/Setting Time (ST) Measurement

The working time of cement paste was determined by the time after that the cement paste was no longer workable. The setting time of cement paste was measured according to the standard method set forth in ISO 1566 for dental zinc phosphate cements. The cement is considered set when a 400 gm weight loaded onto a Vical needle with a 1 mm diameter tip fails to make a perceptible circular indentation on the surface of the cement.

### pH Measurement

The early stage (during setting process) variation in pH was determined using a pH meter (Suntex Instruments SP20004, Taipei, Taiwan) that was buried in the cement paste immediately after the powder and setting liquid were mixed. The first reading was taken at 1 minute after mixing. The measurement was continued until the paste nearly becomes set. Readings were taken every 30 seconds until 30 minutes after mixing. After then they were taken every 60 seconds.

The variation in pH value of Hanks' solution in which the cement paste sample was immersed was monitored using the same pH meter. 2 g cement paste was taken after mixing the powder and the setting solution for 5 minutes, and it was immersed in 20 ml Hanks' solution with a pH value of 7.05 for the test. The solution was maintained at 37° C. throughout testing and continually stirred to help maintain uniform ion concentrations of the solution.

Composition of Hanks' solution (Hench, 1971)

| Component | Concentration (g/L) |
|---|---|
| NaCl | 8.00 |
| Na₂HPO₄ 2H₂O | 0.06 |
| CaCl₂ | 0.14 |
| NaHCO₃ | 0.35 |
| KCl | 0.40 |
| Glucose | 1.00 |
| MgCl₂ 6H₂O | 0.10 |
| MgSO₄ 7H₂O | 0.06 |
| KH₂PO₄ | 0.06 |

### Measurement of Porosity

The porosity of the various samples was measured according to ASTM C830-00 (2006) method, “Standard Test Methods for Apparent Porosity, Liquid Absorption, Apparent Specific Gravity, and Bulk Density of Refractory Shapes by Vacuum Pressure”.

### Results

### Group A—Paste

TABLE A-1
Working time, setting time and CS of calcium sulfate cement prepared
from acidic hardening solution (L/P = 0.35) (n = 6)

| Hardening | Conc. | Solution | | | CS |
|---|---|---|---|---|---|
| solution | (M) | pH | WT (Min) | ST (Min) | (MPa) |
| Water | | 7.0 | 2.0 | 2.4 | 12.7 ± 2.9 |
| H₃PO₄ | 0.01875 | 2.2 | 5.3 | 6:7 | 29.1 ± 1.2 |
| | 0.0375 | 2.1 | 8.5 | 10.3 | 33.7 ± 4.3 |
| | 0.075 | 1.9 | 8.3 | 10.8 | 30.3 ± 2.0 |
| KCl | 0.01875 | 6.7 | 1.3 | 1.5 | 11.5 ± 2.4 |
| | 0.0375 | 6.7 | 1.7 | 1.3 | 6.5 ± 1.7 |
| | 0.075 | 6.9 | 1.8 | 1.4 | 3.8 ± 1.9 |
| HCl | 0.01875 | 2.1 | 2.5 | 2.8 | 16.2 ± 1.6 |
| | 0.0375 | 2.0 | 3.2 | 3.5 | 19.4 ± 1.7 |
| | 0.075 | 1.5 | 3.8 | 4.2 | 22.2 ± 2.4 |
| HNO₃ | 0.01875 | 1.8 | 1.9 | 2.2 | 17.7 ± 2.8 |
| | 0.0375 | 1.5 | 2.5 | 2.8 | 15.0 ± 3.4 |
| | 0.075 | 1.3 | 2.2 | 2:7 | 17.1 ± 1.5 |
| KH₂PO₄ | 0.01875 | 5.1 | 4.6 | 4.9 | 25.5 ± 1.8 |
| | 0.0375 | 4.8 | 5.7 | 6.3 | 23.3 ± 2.9 |
| | 0.075 | 4.6 | 13.4 | 13.8 | 18.7 ± 3.7 |

TABLE A-2
Working time, setting time and CS of calcium sulfate cement prepared
from basic hardening solution (L/P = 0.35) (n = 6)

| Hardening | Conc. | Solution | WT | ST | CS |
|---|---|---|---|---|---|
| solution | (M) | pH | (Min) | (Min) | (MPa) |
| Water | | 7.0 | 2.0 | 2.4 | 12.7 ± 2.9 |
| (NH₄)₂HPO₄ | 0.01875 | 7.8 | 5.5 | 6.3 | 27.5 ± 2.5 |
| | 0.0375 | 7.9 | 8.3 | 10.3 | 33.6 ± 3.6 |
| | 0.075 | 8.0 | 10.2 | 10.8 | 32.0 ± 1.8 |
| K₂HPO₄ | 0.01875 | 8.4 | 4.7 | 5.0 | 28.2 ± 2.9 |
| | 0.0375 | 8.4 | 6.3 | 6.6 | 36.4 ± 2.2 |
| | 0.075 | 9.2 | 5.8 | 6.3 | 33.9 ± 4.0 |
| NaHCO₃ | 0.01875 | 8.5 | 2.9 | 3.5 | 11.6 ± 1.9 |
| | 0.0375 | 8.6 | 3.5 | 4.1 | 11.6 ± 2.2 |
| | 0.075 | 8.6 | 4.3 | 4.8 | 14.2 ± 3.5 |
| NaOH | 0.01875 | 12.1 | 2.4 | 2.5 | 10.1 ± 1.2 |
| | 0.0375 | 12.4 | 2.1 | 2.2 | 9.8 ± 3.4 |
| | 0.075 | 12.7 | 2.0 | 2.1 | 8.8 ± 2.9 |
| NaCl | 0.01875 | 7.2 | 2.0 | 2.5 | 8.6 ± 1.9 |
| | 0.0375 | 7.0 | 1.7 | 2.4 | 7.2 ± 2.7 |
| | 0.075 | 7.5 | 1.7 | 2.3 | 7.0 ± 1.7 |
| Na₂HPO₄ | 0.01875 | 8.4 | 5.8 | 6.3 | 30.9 ± 0.8 |
| | 0.0375 | 8.8 | 5.2 | 6.2 | 34.0 ± 1.3 |
| | 0.075 | 9.4 | 8.3 | 9.2 | 27.2 ± 2.9 |
| Na₂CO₃ | 0.01875 | 10.7 | 2.3 | 2.8 | 18.4 ± 1.3 |
| | 0.0375 | 10.8 | 3.5 | 4.0 | 19.0 ± 2.4 |
| | 0.075 | 11.0 | 2.2 | 2.5 | 16.4 ± 2.0 |

### Summary (Tables A-1 and A-2):

1. All solutions with phosphate (no matter acidic or basic) lead to reasonable WT/ST (about 5-10 min), while all solutions without phosphate (no matter acidic or basic) result in too short WT/ST (for paste injection surgery).

(Note: Too Short Wt/ST Leave Insufficient Time for Preparation and/or Surgery)

2. All solutions with phosphate (no matter acidic or basic) give much higher CS values than those without phosphate.

3. Phosphate concentration of 0.0375 M leads to highest CS value, although CS values from all three concentrations (0.01875-0.075 M) are acceptable.

TABLE A-3
Effect of concentration of (NH₄)₂HPO₄ hardening solution on WT and
ST of calcium sulfate cement

| (NH₄)₂HPO₄ conc. (M) | L/P ratio (cc/g) | WT (Min) | ST (Min) |
|---|---|---|---|
| 0.01875 | 0.35 | 5.5 | 6.3 |
| 0.0375 | 0.35 | 8.3 | 10.3 |
| 0.075 | 0.35 | 10.2 | 10.8 |
| 0.25 | 0.35 | 6.4 | 6.7 |
| 0.50 | 0.35 | 6.7 | 7.0 |
| 0.75 | 0.35 | 7.7 | 8.0 |
| 1.00 | 0.40* | 8.5 | 9.0 |

*L/P ratio is increased to facilitate mixing

TABLE A-4
Effect of concentration of K₂HPO₄ hardening solution on WT and ST
of calcium sulfate cement

| K₂HPO₄ conc. (M) | L/P ratio (cc/g) | WT (Min) | ST (Min) |
|---|---|---|---|
| 0.01875 | 0.35 | 4.7 | 5.0 |
| 0.0375 | 0.35 | 6.3 | 6.6 |
| 0.075 | 0.35 | 5.8 | 6.3 |
| 0.10 | 0.40* | 8.5 | 9.2 |
| 0.25 | 0.40* | 17.5 | 42.0 |

*L/P ratio is increased to facilitate mixing

### Summary (Tables A-3 and A-4):

1. (NH₄)₂HPO₄ solution with phosphate concentration up to 1.0 M gives reasonable WT/ST.

2. K₂HPO₄ solution with phosphate concentration up to 0.1 M gives reasonable WT/ST. When K₂HPO₄ concentration is higher than 0.1 M, WT/ST becomes too long (for paste injection surgery).

(Note: Too long WT/ST indicates a low early strength and easy dispersion of the paste upon contact body fluid/blood prior to hardening)

TABLE A-5
Effect of concentration of (NH₄)₂HPO₄ hardening solution on CS of
calcium sulfate cement (L/P = 0.35) (n = 6)

| Hardening solution | Concentration (M) | CS (MPa) |
|---|---|---|
| Water | | 12.7 ± 2.9 |
| (NH₄)₂HPO₄ | 0.01875 | 27.5 ± 2.5 |
| | 0.0375 | 33.6 ± 3.6 |
| | 0.075 | 32.0 ± 1.8 |
| | 0.10 | 26.8 ± 2.8 |
| | 0.25 | 20.7 ± 4.1 |
| | 0.50 | 17.7 ± 19.0 |
| | 0.75 | 13.3 ± 13.7 |
| | 1.00* | 1.4 ± 1.4 |
| | 2.00* | 3.6 ± 3.7 |

*L/P ratio is increased to 0.40 to facilitate mixing.

### Summary:

1. Phosphate concentration of 0.0375 M leads to highest CS value, although phosphate concentration up to 0.1 M still gives reasonable CS.

2. When phosphate concentration is 0.75 M, the CS value becomes lower than half that of 0.0375 M.

TABLE A-6
Effect of pH value on WT and ST of calcium sulfate cement (n = 6)

| Hardening | | | | |
|---|---|---|---|---|
| solution | pH value | L/P (cc/g) | WT (Min) | ST (Min) |
| Water | 7.0 | 0.40 | 2.0 | 2.4 |
| (NH₄)₂HPO₄ | 1.0 | 0.35 | 5.3 | 6.7 |
| | 3.0 | 0.35 | 7.8 | 8.2 |
| | 5.0 | 0.35 | 9.8 | 10.3 |
| | 7.9 | 0.35 | 8.3 | 10.3 |
| | 9.0 | 0.35 | 10.3 | 11.0 |
| | 11.0 | 0.40 | 3.3 | 3.7 |
| | 13.0 | 0.40 | 1.7 | 2.2 |
| K₂HPO₄ | 8.4 | 0.35 | 6.3 | 6.6 |
| | 9.0 | 0.35 | 5.8 | 6.3 |
| | 11.0 | 0.35 | 5.5 | 5.8 |
| | 13.0 | 0.40 | 2.3 | 2.5 |

### Summary:

1. WT/ST values are very sensitive to the pH value of the hardening solution.

2. When phosphate-containing solution (no matter (NH₄)₂HPO₄) or K₂HPO₄) has a pH value higher than about 11, WT/ST largely decreases to become unacceptably short.

TABLE A-7
Effect of pH value on CS of calcium sulfate cement

| Hardening solution | pH value | L/P (cc/g) | CS (MPa) |
|---|---|---|---|
| Water | 7.0 | 0.40 | 12.7 ± 2.9 |
| (NH₄)₂HPO₄ | 1.0 | 0.35 | 30.4 ± 2.0 |
| | 3.0 | 0.35 | 31.2 ± 3.4 |
| | 5.0 | 0.35 | 29.9 ± 3.1 |
| | 7.9 | 0.35 | 33.6 ± 3.6 |
| | 9.0 | 0.35 | 32.1 ± 2.1 |
| | 11.0 | 0.40 | 22.9 ± 4.0 |
| | 13.0 | 0.40 | 10.8 ± 1.6 |
| K₂HPO₄ | 1.0 | 0.35 | 19.5 ± 1.0 |
| | 3.0 | 0.35 | 23.7 ± 2.4 |
| | 5.0 | 0.35 | 24.9 ± 0.8 |
| | 8.4 | 0.35 | 36.4 ± 2.2 |
| | 11.0 | 0.35 | 36.2 ± 1.3 |
| | 12.0 | 0.35 | 30.0 ± 4.6 |
| | 13.0 | 0.40 | 11.9 ± 1.4 |

[Note:
(NH₄)₂HPO₄ conc: 0.0375M, pH = 7.9; K₂HPO₄ conc. 0.0375M, pH = 8.4. Adding HCl to decrease pH value, while adding NaOH to increase pH value) (n = 6)

### Summary:

1. CS values are very sensitive to the pH value of the hardening solution.

2. When (NH₄)₂HPO₄ hardening solution has a pH value higher than about 11, the CS value largely decreases. When its pH value reaches 13.0, the CS value decreases by 68% from its highest CS.

3. When K₂HPO₄ has a pH value higher than about 12.0, the CS value largely decreases. When its pH value reaches 13.0, the CS value decreases by 67% from its highest CS.

TABLE A-8
Early stage pH variation of calcium sulfate cement paste
prepared from 0.0375M (NH₄)₂HPO₄ hardening solution

| Time (min) | 2.5 | 5.0 | 7.5 | 10.0 | 12.5 | 15.0 |
|---|---|---|---|---|---|---|
| Paste pH | 6.2 | 6.4 | 6.6 | 6.7 | 6.7 | 6.8 |

### Summary:

The pH value of the paste (mixing CSH and 0.0375M (NH₄)₂HPO₄) slowly increases to a range between 6.5 and 7.0 after mixing for 15 min, a range acceptable to tissues/cells.

TABLE A-9
pH variation of Hanks' solution wherein calcium sulfate cement
prepared from 0.0375M (NH₄)₂HPO₄ hardening solution is immersed.

| | Immersion time (day) | | | | |
|---|---|---|---|---|---|
| | 1 | 3 | 7 | 14 | 40 |
| Hanks' | 6.2 ± 0.5 | 6.0 ± 0.2 | 6.3 ± 0.1 | 6.6 ± 0.1 | 6.6 ± 0.3 |
| solution pH | | | | | |

### Summary:

The pH value of the Hanks' solution is always between 6 and 7, a range acceptable to tissues/cells. After 14 days, it becomes close to neutral.

TABLE A-10
CS variation of calcium sulfate cement prepared from de-ionized water,
0.0375M (NH₄)₂HPO₄ and 0.0375M K₂HPO₄ hardening solutions
followed by immersion in Hanks' solution for different periods of time
(n = 6).

| Hardening solution | Conc. (M) | Time (day) | CS (MPa) |
|---|---|---|---|
| De-ionized water | | 1 | 12.7 ± 2.9 |
| | | 3 | 9.4 ± 3.0 |
| | | 7 | 11.1 ± 2.2 |
| | | 14 | 9.1 ± 3.8 |
| | | 30 | 6.0 ± 2.0 |
| (NH₄)₂HPO₄ | 0.0375 | 1 | 33.6 ± 3.6 |
| | | 3 | 29.6 ± 1.2 |
| | | 7 | 30.0 ± 2.1 |
| | | 14 | 25.1 ± 4.1 |
| | | 30 | 26.4 ± 3.2 |
| K₂HPO₄ | 0.0375 | 1 | 36.4 ± 2.2 |
| | | 3 | 24.1 ± 1.8 |
| | | 7 | 18.8 ± 4.2 |
| | | 14 | 19.0 ± 2.0 |
| | | 30 | 16.9 ± 1.6 |

### Summary:

1. The CS values of calcium sulfate cement prepared from de-ionized water are far lower than from other two phosphate-containing hardening solutions.

2. The CS values of calcium sulfate cement prepared from phosphate-containing hardening solutions decay reasonably slowly when stored in Hanks' solution for a long period of time.

3. The calcium sulfate cement prepared from (NH₄)₂HPO₄ hardening solution behaves exceptionally well in terms of maintaining CS value when stored in Hanks' solution. After 30 days, its CS value decreases only by less than 20%, compared to 1-day value.

TABLE A-11
Effect of tartaric acid addition on CS of calcium sulfate cement
prepared from 0.0375M (NH₄)₂HPO₄ hardening solution

Hardening solution

| | 0.0375M | 0.0375M (NH₄)₂HPO₄/tartaric |
|---|---|---|
| | (NH₄)₂HPO₄ | acid (1:1 by wt) |
| CS (MPa) | 26.5 | 31.3 |

### Summary:

1. The CS value of calcium sulfate cement prepared from (NH₄)₂HPO₄/tartaric acid hardening solution is higher than that prepared from (NH₄)₂HPO₄ by 18%.

### Group B—Dense Block

[NOTE: For pre-formed block samples, “WT/ST” is no longer an issue and K₂HPO₄ was selected as hardening solution for study]

TABLE B-1
Effect of K₂HPO₄ hardening solution concentration on CS of calcium
sulfate dense block article (L/P = 0.35)

| K₂HPO₄ conc. (M) | CS (MPa) |
|---|---|
| 0.01875 | 97.1 ± 2.6 |
| 0.0375 | 100.2 ± 5.9 |
| 0.056 | 108.2 ± 7.8 |
| 0.075 | 113.5 ± 8.7 |
| 0.10 | 106.3 ± 4.1 |
| 0.15 | 101.6 ± 4.8 |
| 0.20 | 63.6 ± 7.1 |
| 0.40 | 21.7 ± 1.7 |
| 1.00 | 23.8 ± 1.3 |

### Summary:

1. Calcium sulfate dense block samples prepared from K₂HPO₄ hardening solution with phosphate concentrations from 0.01875 M to 0.15 M have high CS values. When the concentration is higher than 0.20 M, CS largely decreases to unacceptably low values.

2. Concentration of 0.075 M results in the highest CS value.

TABLE B-2
CS values of calcium sulfate dense block samples prepared from 0.075M
K₂HPO₄ hardening solution (L/P = 0.35) followed by impregnating
treatment in different Impregnating solutions for 1 day. (n = 6)

| Impregnating solution | Impregnating temperature (° C.) | CS (MPa) |
|---|---|---|
| None | | 113.5 ± 8.7 |
| Hanks' solution | 0 | 111.9 ± 4.2 |
| | 37 | 100.4 ± 3.0 |
| K₂HPO₄ | 0 | 129.4 ± 8.3 |
| 1M | 37 | 117.0 ± 11.6 |
| NaOH | 0 | 81.4 ± 7.4 |
| 1M | 37 | 78.7 ± 6.8 |
| KCl | 0 | 74.6 ± 9.5 |
| 3M | 37 | 74.0 ± 7.0 |
| HNO₃ | 0 | 3.2 ± 1.2 |
| 15.4M | 37 | 6.0 ± 0.5 |

### Summary:

1. Dense block samples prepared from 0.075M K₂HPO₄ hardening solutions have much higher CS values after impregnating for 1 day in phosphate-containing solutions than in solutions without phosphate. [Note: HANKS' solution is a phosphate-containing solution]

2. Lower impregnation temperature (0° C.) results in higher CS than higher impregnation temperature (37° C.), no matter the impregnating solution is Hanks' solution or K₂HPO₄ solution.

TABLE B-3
CS variation of calcium sulfate dense block prepared from 0.075M
K₂HPO₄ hardening solution (L/P = 0.35) followed by immersion in
Hanks' solution for different periods of time. (n = 6)

| Hardening solution | Conc. (M) | Immersion time (day) | CS (MPa) |
|---|---|---|---|
| K₂HPO₄ | 0.0375 | None | 92.1 ± 1.1 |
| | | 1 | 88.6 ± 3.7 |
| | | 4 | 77.7 ± 3.1 |
| | | 8 | 76.0 ± 3.0 |
| | | 16 | 70.5 ± 1.3 |
| | 0.075 | None | 113.5 ± 8.7 |
| | | 1 | 99.7 ± 3.0 |
| | | 4 | 87.3 ± 1.2 |
| | | 8 | 86.2 ± 3.0 |
| | | 16 | 78.7 ± 2.7 |

### Summary:

1. Dense block samples prepared from K₂HPO₄ hardening solution (no matter the concentration is 0.0375 M or 0.075M) decay reasonably slowly in CS when immersed in Hanks' solution. After immersion for 16 days, they still maintain about 70% of their original CS values.

### Group C—Porous Block

### Preparation of Calcium Sulfate Porous Block Samples

The calcium sulfate porous block samples for the study were prepared by first mixing CSH and KCl powders (1:1 by weight) with 0.075 M K₂HPO₄ hardening solution at a L/P ratio of 0.35 cc/g) to form a KCl/CSH cement paste. Prior to being fully hardened, the KCl/CSH cement paste was placed in a mold under a pressure of 450 Kgf to squeeze a portion of the hardening solution out of the paste to form a hardened dense block. After being removed from the mold, the hardened dense block sample was immersed in de-ionized water for 3 days to allow the pore-forming particles to be washed out of the dense block to form a calcium sulfate porous block, followed by drying in an oven at 50° C. for 1 day. The X-ray diffraction results indicated that the KCl phase was totally dissolved (KCl peaks disappeared from the XRD patterns) after immersion in de-ionized water at 37 C or 4 C for 3 days.

One group of the porous block samples was further impregnated in an impregnation solution ((NH₄)₂HPO₄ or K₂HPO₄) at 37° C. or 4° C. for a period of time to allow the strength of the porous block to increase, followed by drying in an oven at 50° C. for 1 day.

Another group of samples was further treated by immersion in CaCl₂ solution for 1-3 days to further enhance the strength of the porous samples.

TABLE C-1
CS values of calcium sulfate porous block samples prepared by
immersion for 3 days in de-ionized water at 37° C. and 4° C. to
remove KCl particles.

Immersion
temperature (° C.)

| | 37 | 4 |
|---|---|---|
| CS (MPa) | 2.8 | 4.8 |

### Summary:

1. The CS value of the porous block sample immersed in 4° C. de-ionized water (4.8 MPa) is higher than that immersed in 37° C. de-ionized water (2.8 MPa) by 71%.

TABLE C-2
CS values of impregnation-treated porous block samples under
different impregnating conditions. (The KCl particles were dissolved by
immersion in 4° C. de-ionized water for 3 days)

| Impregnating solution | Solution conc. | Impregnating | |
|---|---|---|---|
| (4° C.) | (M) | time (day) | CS (MPa) |
| None | — | — | 4.8 ± 0.7 |
| (NH₄)₂HPO₄ | 1.0 | 1 | 4.7 ± 0.8 |
| (NH₄)₂HPO₄ | 1.5 | 1 | 5.5 ± 1.2 |
| (NH₄)₂HPO₄ | 2.0 | 1 | 7.0 ± 1.8 |
| K₂HPO₄ | 1.0 | 1 | 4.5 ± 1.1 |
| K₂HPO₄ | 1.0 | 2 | 5.0 ± 1.4 |
| K₂HPO₄ | 1.0 | 3 | 4.3 ± 1.2 |
| K₂HPO₄ | 1.5 | 1 | 5.4 ± 1.1 |
| K₂HPO₄ | 1.5 | 2 | 5.4 ± 0.9 |
| K₂HPO₄ | 1.5 | 3 | 5.9 ± 1.1 |
| K₂HPO₄ | 2.0 | 1 | 6.2 ± 1.0 |
| K₂HPO₄ | 2.0 | 3 | 10.2 ± 2.0 |

### Summary:

1. In both impregnating solutions, CS values largely increase when the concentration of the impregnating solution increases.

2. After impregnating for 1 day, the CS increases by 46% in 2M (NH₄)₂HPO₄ and 29% in 2M K₂HPO₄ impregnating solution. After impregnating for 3 days, the CS increases by as much as 113% in 2M K₂HPO₄ impregnating solution.

### CaCl₂ Treatment to Further Enhance CS

To further enhance the strength of the porous block sample, a calcium sulfate porous block sample with a CS value of 5.0 MPa (control) was immersed in CaCl₂ solution under different conditions

TABLE C-3
CS values of calcium sulfate porous block samples treated by immersion
in CaCl₂ solution under different conditions.

| CaCl₂ solution conc. (M) | Immersion time (day) | CS (MPa) |
|---|---|---|
| Control | — | 5.0 ± 0.2 |
| 1.0 | 1 | 13.7 ± 1.7 |
| | 3 | 12.2 ± 1.3 |
| | 5 | 9.6 ± 0.7 |
| | 7 | 8.0 ± 0.5 |
| 2.0 | 1 | 14.9 ± 1.6 |
| | 3 | 21.1 ± 2.5 |
| | 5 | 13.7 ± 1.0 |
| | 7 | 11.0 ± 1.0 |
| 3.0 | 1 | 17.3 ± 1.9 |
| | 3 | 25.1 ± 2.6 |
| | 5 | 17.0 ± 2.3 |
| | 7 | 17.4 ± 1.0 |
| 4.0 | 1 | 14.0 ± 1.9 |
| | 3 | 13.3 ± 2.9 |
| | 5 | 14.8 ± 2.0 |
| | 7 | 17.3 ± 3.6 |

### Results:

1. After CaCl₂ treatment, the CS largely increases under all conditions.

2. The largest increases in CS were found in the treatments of 2M CaCl₂ for 3 days (from 5.0 MPa to 21.1 MPa, an increase of 322%) and 3M CaCl₂ for 3 days (from 5.0 MPa to 25.1 MPa, an increase of 402%)

1. A method for preparing a hardened calcium sulfate dihydrate block comprising mixing calcium sulfate hemihydrate powder with an aqueous solution containing phosphate ions so as to form a paste, wherein the aqueous solution has a pH value lower than 10.

2. The method of claim 1 wherein there in no alkaline compound being added to the aqueous solution, the calcium sulfate hemihydrate power or the paste thereof to adjust pH values thereof.

3. The method of claim 1 wherein the aqueous solution or the calcium sulfate hemihydrate powder has a temperature lower than 50° C. before said mixing.

4. The method of claim 3 wherein said mixing is conducted at a temperature lower than 50° C.

5. The method of claim 3 wherein the paste of the power and the aqueous solution has a temperature lower than 50° C.

6. The method of claim 1 wherein there is no heating being applied to the aqueous solution, the calcium sulfate hemihydrate power or the paste thereof to elevate temperatures thereof to equal to or greater than 50° C.

7. The method of claim 6 wherein the aqueous solution has a concentration of the phosphate ions lower than 1.0 M.

8. The method of claim 7, wherein the concentration is of 0.01 M to 0.5 M.

9. The method of claim 1 wherein the mixing is conducted with a liquid to powder ratio of 0.20 cc/g to 0.60 cc/g.

10. The method of claim 9, wherein the liquid to powder ratio is 0.30 cc/g to 0.50 cc/g.

11. The method of claim 1, wherein said aqueous solution is an aqueous solution of (NH₄)₃PO₄, (NH₄)₂HPO₄, NH₄H₂PO₄, K₃PO₄, K₂HPO₄, KH₂PO₄, Na₃PO₄, Na₂HPO₄, NaH₂PO₄, H₃PO₄, or a mixture thereof.

12. The method of claim 11, wherein said aqueous solution is an aqueous solution of (NH₄)₃PO₄, (NH₄)₂HPO₄, NH₄H₂PO₄, or a mixture thereof.

13. The method of claim 1 where in said aqueous solution or said calcium sulfate hemihydrate powder further comprises living cells, a growth factor or a drug.

14. The method of claim 1, wherein said calcium sulfate hemihydrate powder is an alpha-type calcium sulfate hemihydrate powder.

15. The method of claim 1 further comprising introducing the paste to a hole or cavity and letting the paste become set in-situ to form a block of hardened calcium sulfate dihydrate in the hole or cavity.

16. The method of claim 1 further comprising shaping the paste in a mold, and removing the mold to form a block of hardened calcium sulfate dihydrate.

17. The method of claim 16 further comprising pressurizing said paste in said mold before said paste becomes set to remove a portion of liquid from said paste, so that a liquid to powder ratio of said paste decreases.

18. The method of claim 17, wherein the pressure applied to the paste in the mold is form about 1 MPa to 500 MPa, preferably from 100 MPa to 500 MPa.

19. The method of claim 16 further comprising impregnating the block with an impregnating liquid for a period of time, so that a compressive strength of the resulting impregnated block removed from the impregnating liquid is increased compared to that of said block without said impregnating treatment.

20. The method of claim 19 wherein the impregnating liquid is a phosphate-containing solution.

21. The method of claim 20, wherein the phosphate-containing solution is an aqueous solution of (NH₄)₃PO₄, (NH₄)₂HPO₄, NH₄H₂PO₄, K₃PO₄, K₂HPO₄, KH₂PO₄, Na₃PO₄, Na₂HPO₄, NaH₂PO₄, or H₃PO₄.

22. The method of claim 20, wherein said phosphate-containing solution has a phosphate concentration from about 0.1 M to about 6 M, preferably from about 1 M to about 3 M.

23. The method of claim 20, wherein said impregnating is conducted at a temperature of about 0° C.

24. The method of claim 1 further comprising mixing a pore-forming agent with the powder or with the paste; shaping the paste in a mold; removing the mold to form a block of hardened calcium sulfate dihydrate with the pore-forming agent embedded therein; and immersing said block of hardened calcium sulfate dihydrate with the pore-forming agent embedded therein in an immersing liquid to dissolve said pore-forming agent in the immersing liquid, creating pores therein, so that a porous block is formed.

25. The method of claim 24, wherein the pore forming agent is selected from the group consisting of LiCl, KCl, NaCl, MgCl₂, CaCl₂, NaIO₃, KI, Na₃PO₄, K₃PO₄, Na₂CO₃, amino acid-sodium salt, amino acid-potassium salt, glucose, polysaccharide, fatty acid-sodium salt, fatty acid-potassium salt, potassium bitartrate (KHC₄H₄O₆), potassium carbonate, potassium gluconate (KC₆H₁₁O₇), potassium-sodium tartrate (KNaC₄H₄O₆.4H₂O), potassium sulfate (K₂SO₄), sodium sulfate, sodium lactate and mannitol.

26. The method of claim 24, wherein the immersing liquid is an acidic aqueous solution, a basic aqueous solution, a physiological solution, an organic solvent, or water.

27. The method of claim 26, wherein the immersing liquid is a phosphate-containing solution having a phosphate concentration from about 0.1 M to about 6 M, preferably from about 1 M to about 3 M.

28. The method of claim 27, wherein the phosphate-containing solution is an aqueous solution of (NH₄)₃PO₄, (NH₄)₂HPO₄, NH₄H₂PO₄, K₃PO₄, K₂HPO₄, KH₂PO₄, Na₃PO₄, Na₂HPO₄, NaH₂PO₄, or H₃PO₄.

29. The method of claim 27, wherein the immersing liquid is water.

30. The method of claim 24 further comprising impregnating the porous block with an impregnating liquid for a period of time, so that a compressive strength of the resulting impregnated porous block removed from the impregnating liquid is increased compared to that of said porous block without said impregnating treatment.

31. The method of claim 30 wherein the impregnating liquid is a phosphate-containing solution.

32. The method of claim 31, wherein the phosphate-containing solution is an aqueous solution of (NH₄)₃PO₄, (NH₄)₂HPO₄, NH₄H₂PO₄, K₃PO₄, K₂HPO₄, KH₂PO₄, Na₃PO₄, Na₂HPO₄, NaH₂PO₄, or H₃PO₄.

33. The method of claim 31, wherein said phosphate-containing solution has a phosphate concentration from about 0.1 M to about 6 M, preferably from about 1 M to about 3 M.

34. The method of claim 27 further comprising impregnating the porous block in a suspension of living cells or a solution of growth factor or drug to deposit the living cells, the growth factor or drug onto the porous block.

35. The method of claim 24, wherein the porous block has a porosity of 50-90 vol %.

36. A method for treating a subject comprising mixing calcium sulfate hemihydrate powder with an aqueous solution containing phosphate ions so as to form a paste; and filling a hole or cavity in a bone of said subject with said paste which set hard in the hole or cavity in need of said treatment.

37. The method of claim 36 wherein said treatment is an orthopedic treatment or a dental treatment.

38. A method for treating a subject comprising mixing calcium sulfate hemihydrate powder with an aqueous solution containing phosphate ions so as to form a paste; forming a block of hardened calcium sulfate dihydrate from said paste; and implanting said block in said subject in need of said treatment.

39. The method of claim 38 wherein said treatment is an orthopedic treatment or a dental treatment.

40. The method of claim 39 wherein said implanting comprises breaking up the block into pellets and filling a hole or cavity in a bone of said subject with said pellets.

41. The method of claim 40 wherein said formation of the block of hardened calcium sulfate dihydrate comprising shaping the paste in a mold, and removing the mold to form a block of hardened calcium sulfate dihydrate.

42. The method of claim 41 wherein said formation of the block of hardened calcium sulfate dihydrate further comprising pressurizing said paste in said mold before said paste becomes set to remove a portion of liquid from said paste, so that a liquid to powder ratio of said paste decreases.

43. The method of claim 42, wherein the pressure applied to the paste in the mold is form about 1 MPa to 500 MPa, preferably from 100 MPa to 500 MPa.

44. The method of claim 41, wherein said formation of the block of hardened calcium sulfate dihydrate further comprising impregnating the block with an impregnating liquid for a period of time, so that a compressive strength of the resulting impregnated block removed from the impregnating liquid is increased compared to that of said block without said impregnating treatment.

45. The method of claim 44 wherein the impregnating liquid is a phosphate-containing solution.

46. The method of claim 45, wherein the phosphate-containing solution is an aqueous solution of (NH₄)₃PO₄, (NH₄)₂HPO₄, NH₄H₂PO₄, K₃PO₄, K₂HPO₄, KH₂PO₄, Na₃PO₄, Na₂HPO₄, NaH₂PO₄, or H₃PO₄.

47. The method of claim 45, wherein said phosphate-containing solution has a phosphate concentration from about 0.1 M to about 6 M, preferably from about 1 M to about 3 M.

48. The method of claim 45, wherein said impregnating is conducted at a temperature of about 0° C.

49. The method of claim 38 further comprising mixing a pore-forming agent with the powder or with the paste, wherein said formation of the block of hardened calcium sulfate dihydrate comprises shaping the paste in a mold; removing the mold to form a block of hardened calcium sulfate dihydrate with the pore-forming agent embedded therein; and immersing said block of hardened calcium sulfate dihydrate with the pore-forming agent embedded therein in an immersing liquid to dissolve said pore-forming agent in the immersing liquid, creating pores therein, so that a porous block is formed, and thus said implanting comprises implanting said porous block in said subject in need of said treatment.

50. The method of claim 49 wherein said implanting comprises breaking up the porous block into pellets and filling a hole or cavity in a bone of said subject with said pellets.

51. The method of claim 50, wherein the pore forming agent is selected from the group consisting of LiCl, KCl, NaCl, MgCl₂, CaCl₂, NaIO₃, KI, Na₃PO₄, K₃PO₄, Na₂CO₃, amino acid-sodium salt, amino acid-potassium salt, glucose, polysaccharide, fatty acid-sodium salt, fatty acid-potassium salt, potassium bitartrate (KHC₄H₄O₆), potassium carbonate, potassium gluconate (KC₆H₁₁O₇), potassium-sodium tartrate (KNaC₄H₄O₆.4H₂O), potassium sulfate (K₂SO₄), sodium sulfate, sodium lactate and mannitol.

52. The method of claim 50, wherein the immersing liquid is an acidic aqueous solution, a basic aqueous solution, a physiological solution, an organic solvent, or water.

53. The method of claim 52, wherein the immersing liquid is a phosphate-containing solution having a phosphate concentration from about 0.1 M to about 6 M, preferably from about 1 M to about 3 M.

54. The method of claim 53, wherein the phosphate-containing solution is an aqueous solution of (NH₄)₃PO₄, (NH₄)₂HPO₄, NH₄H₂PO₄, K₃PO₄, K₂HPO₄, KH₂PO₄, Na₃PO₄, Na₂HPO₄, NaH₂PO₄, or H₃PO₄.

55. The method of claim 53, wherein said immersing liquid is water.

56. The method of claim 50 further comprising impregnating the porous block with an impregnating liquid for a period of time, so that a compressive strength of the resulting impregnated porous block removed from the impregnating liquid is increased compared to that of said porous block without said impregnating treatment.

57. The method of claim 56 wherein the impregnating liquid is a phosphate-containing solution.

58. The method of claim 57, wherein the phosphate-containing solution is an aqueous solution of (NH₄)₃PO₄, (NH₄)₂HPO₄, NH₄H₂PO₄, K₃PO₄, K₂HPO₄, KH₂PO₄, Na₃PO₄, Na₂HPO₄, NaH₂PO₄, or H₃PO₄.

59. The method of claim 57, wherein said phosphate-containing solution has a phosphate concentration from about 0.1 M to about 6 M, preferably from about 1 M to about 3 M.

60. The method of claim 49 further comprising impregnating the porous block in a suspension of living cells or a solution of growth factor or drug to deposit the living cells, the growth factor or drug onto the porous block.

61. The method of claim 49, wherein the porous block has a porosity of 50-90 vol %.

62. A bone cement formula composed of calcium sulfate hemihydrate powder, an aqueous solution containing phosphate ions, and optionally one or more functional components.

63. The formula of claim 62 wherein the one or more functional components are living cells, a growth factor, a drug or a pore forming agent.
